(19) United States
(12) Patent Application Publication
(10) Pub. No.: US 2003/0013905 A1
(43) Pub. Date: Jan. 16, 2003

(54) Salts of cis-4-cyano-4[3(cyclopentyloxy)-4-methoxyphenyl]cyclohexane-1-carboxylic acid

(76) Inventor: Guishu Kris Huang, Audubon, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/149,524

(22) PCT Filed: Dec. 15, 2000
(86) PCT No.: PCT/US00/33966

(51) Int. Cl. C07C 255/45

(52) U.S. Cl. 558/430

(57) ABSTRACT

Salts of Cis-4-cyano-4-[3-(cyclopentyloxy)-4-methoxyphenyl]cyclohexane-1-carboxylate are disclosed herein.

### AREA OF THE INVENTION

This invention relates to certain salts of a carboxylic acid useful for treating diseases treatable by inhibiting the PDE4 enzyme.

### BACKGROUND OF THE INVENTION

Cyclic nucleotide phosphodiesterases (PDEs) represent a family of enzymes that hydrolyze the ubiquitous intracellular second messengers, adenosine 3′,5′-monophosphate (cAMP) and guanosine 3′,5′-monophosphate (cGMP) to their corresponding inactive 5′-monophosphate metabolites. At least ten distinct classes of PDE isozymes are believed to exist, each possessing unique physical and kinetic characteristics and each representing a product of a different gene family. These are distinguished using Arabic numerals 1-10.

The enzyme targeted in this invention is the PDE4 isozyme in all its various forms and in the full domain of its distributions in all cells. It is a low Kₘ (cAMP Kₘ=1-5 μM) cAMP-selective enzyme that has little activity against cGMP (Km>100 μM).

Current PDE inhibitors used in treating inflammation and as bronchodilators, drugs like theophylline and pentoxyfyllin, inhibit PDE isozymes indiscriminately in all tissues. These compounds exhibit side effects, apparently because they non-selectively inhibit all PDE isozyme classes in all tissues. The targeted disease state may be effectively treated by such compounds, but unwanted secondary effects may be exhibited which, if they could be avoided or minimized, would increase the overall therapeutic effect of this approach to treating certain disease states.

A new approach toward improving the side effect profile of PDE inhibitors is to design a new generation of compounds that inhibit only a single PDE isozyme, i.e., the PDE isozyme that predominates in the tissue of cell of interest. The predominate cAMP PDE isozyme in immune and inflammatory cells is PDE4. It is also a major regulator of cAMP content in airway smooth muscle. Thus, selective inhibition of PDE4 elevates cAMP content in immune and inflammatory cells, as well as in airway smooth muscle. This leads to anti-inflammatory effects as well as bronchodilation. One or both of these therapeutic actions are useful in treating a variety of diseases, including, but not limited to asthma and COPD. PDE4 inhibitors, particularly PDE4-specific inhibitors are useful also in treating other diseases in the area of inflammation, (e.g., asthma, chronic obstructive pulmonary disease, inflammatory bowel disease, rheumatoid arthritis), affects related to tumor necrosis factor and to cognition impairment (e.g., multi-infarct dementia, cognitive dysfunction, or stroke).

Although in theory isozyme-selective PDE inhibitors should represent an improvement over non-selective inhibitors, the selective inhibitors tested to date are not devoid of side effects produced as an extension of inhibiting the isozyme of interest in an inappropriate or untargeted tissue, or because they may have cross-reactivity with other PDE isozymes. For example, clinical studies with the selective PDE4 inhibitor rolipram, which was being developed as an antidepressant, indicate it has psychotropic activity and produces gastrointestinal effects, e.g., pyrosis, nausea and emesis. Indications are that side effects of denbufylline, another PDE4 inhibitor targeted for the treatment of multi-infarct dementia, may include pyrosis, nausea and emesis as well. These side effects are thought to occur as a result of inhibiting PDE4 in specific areas of the central nervous system and the gastrointestinal system.

While to date no one has been able to identify a compound which is completely without unwanted side effects at all possible dosage levels, at least one compound has been identified that appears to be better tolerated than previous PDE4 inhibitors, namely cis-4-cyano-4-[3- (cyclopentyloxy)-4-methoxyphenyl]cyclohexane-1-carboxylic acid. Ariflo® is the registered trademark for this compound. This invention provides unique and useful salts of this acid.

### SUMMARY OF THE INVENTION

This invention relates to the following salts of cis-4-cyano-4-[3-(cyclopentyloxy)-4-methoxyphenyl]cyclohexane-1-carboxylic acid: sodium, ethylene diamine and tris(hydroxymethy)aminomethane.

This invention also relates to methods for making these salts

### DETAILED DESCRIPTION OF THE INVENTION

The preparation of cis-4-cyano-4-[3- (cyclopentyloxy)-4-methoxyphenyl]cyclohexane-1-carboxylic acid is described in several publications, for example U.S. Pat. No. 5,552,438 issued Sep. 03, 1996 and PCT application number PCT/US98/02749 and published on Aug. 13, 1998 as WO 98/34584.

Salts of this acid were prepared and characterized as per the following examples.

### EXAMPLE 1

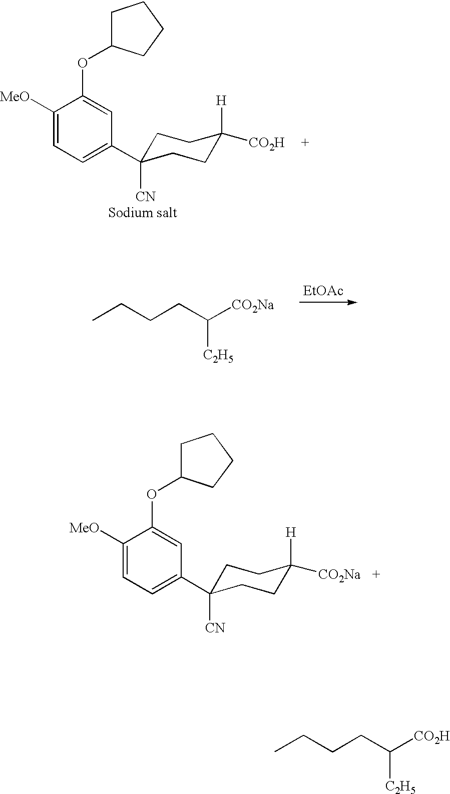

1(a) A 50 ml 3 necked round bottom flask equipped with thermometer, reflux condenser, addition funnel and N₂ inlet was charged with 2.01 g (5.85 mmoles) of cis-4-cyano-4-[3- (cyclopentyloxy)-4-methoxyphenyl]cyclohexane-1-carboxylic acid and 20 ml of ethyl acetate at room temperature under N₂ atmosphere. The resulting mixture was warmed up to 55-60° C. over a period of 20 minutes to obtain clear colorless solution. To this solution was added a pre-made solution of 1.16 g (7.03 mmoles, 1.2 equivalents) of sodium hexanoate in 8.0 ml ethyl acetate slowly over 2 minutes period while maintaining the reaction temperature between 50-60° C. The contents of the reaction flask were cooled to room temperature (18-20° C.) over a period of 30 minutes; precipitation started at around 25-30° C. The suspension was stirred at room temperature for one hour. The contents of the reaction vessel were filtered through a sintered funnel and the cake was rinsed with 2×10 ml of ethyl acetate. This crude product was dried under Hi-vac for 24 hours to a constant weight.

M. P.: 238° C. Karl Fisher: 1.64%.

¹H NMR was satisfactory.

DSC indicated three small endotherms at 154.4° C., 185.1° C. and 212.0° C., one major endotherm at 230° C. and probable decomposition at 324.3° C.

IR(KBr pellet): 3420, 2953, 2232, 1559, 1519, 1415, 1260, 1241, 1167, 1148, 1027, 992, 805 cm⁻¹.

1(b) Recrystallization: A 25 ml 3 neck round bottom flask was equipped with thermometer, reflux condenser, addition funnel and an N₂ inlet, and then charged with 0.606 g (1.654 mmoles) of crude cis-4-cyano-4-[3-(cyclopentyloxy)-4-methoxyphenyl]cyclohexane-1-carboxylic acid sodium salt and 4.5 ml of acetonitrile at room temperature with stirring. To this thick suspension was added deionized water dropwise with heating up to 60° C., with total of 0.3 ml water added. The mixture turned to a clear pale yellow solution. It was air cooled to 45° C., seeded with 2 mg of seed crystal and cooled further to 35° C. over 5 minutes. Some cloudiness was noticed. It was cooled further with an ice-water bath to 0° C. with vigorous stirring. More precipitation formed. The suspension was placed in the freezer (−8° C.) for 16 hours. The contents of reaction flask were filtered through sintered funnel and the filter cake was rinsed with 2×1.5 ml of acetonitrile. The product was dried under Hi-vac for 24 hours to constant weight.

M. P.: >240° C. (turned to brown tar at 260° C.).

DSC showed two small endotherms at 162.1° C. and 189.2° C. and one major endotherm at 232.7° C., followed by probable decomposition at 333.0° C.

IR(KBr) was identical to the crude product.

Karl Fisher: 2.9%; this material upon exposed to air at room temperature for 1 week had 22.9% water content on Karl Fisher, indicating fairly high hydroscopic nature of this salt.

Recrystallization from isopropanol gave similar crystal form (in terms of M. P., DSC, KF, IR and ¹H NMR).

### EXAMPLE 2

### Ethylene Diamine Salt

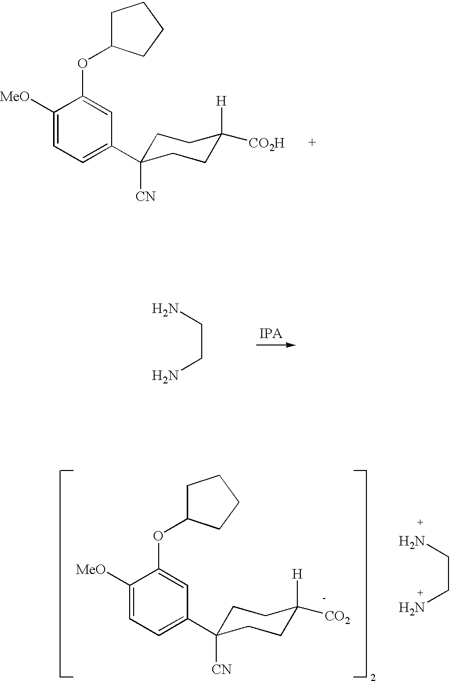

2(a) A 100 ml 3 neck round bottom flask was equipped with a reflux condenser, thermometer, magnetic stirrer and an N₂ inlet. This flask was charged with 6.15 g (16.51 mmoles) of cis-4-cyano-4-[3- (cyclopentyloxy)-4-methoxyphenyl]cyclohexane-1-carboxylic acid and 55 ml of isopropanol at room temperature under N₂ atmosphere. The resulting suspension was heated up to 60° C. with stirring giving a clear light brown solution. To this mixture was added 0.60 ml (0.54 g, 8.96 mmoles, 0.51 equivalents ) of ethylene diamine in one portion. The resulting solution was kept at 60° C. for 15 minutes. The mixture was allowed to cool to 45° C. over 20 minutes and seeded with 2 mg of seed crystal. In few second the salt started to precipitate. The cooling was continued to room temperature then to 0° C. over 45 minutes. The suspension was stirred at 0° C. for additional 2 hours then was filtered through sintered funnel. The filter cake was rinsed with 2×2.0 ml cold isopropanol. The product was dried under Hi-Vac for 20 hours to a constant weight.

¹H NMR was satisfactory; M. P.: 158-161° C.; Karl Fisher: 0.15%;

DSC one small endotherm at 77.4° C., one major at 167.4° C. and probable decomposition at >180° C.; IR(KBr): 3379, 2959, 2860, 2638, 2229, 1617, 1519, 1414, 1268, 1234, 1148, 1024, 988, 936, 807, 739, 634 cm⁻¹.

2(b) Recrystallization: A 25 ml 3 neck round bottom flask equipped with thermometer, reflux condenser, addition funnel and N₂ inlet was charged with 1.02 g of crude ethylene diamine salt (2.74 mmoles) and 12.0 ml of isopropanol at room temperature (18-20° C.). The resulting mixture was heated up to reflux at 90-92° C. over a period of 30 minutes to obtain a clear brown solution. The heating was removed and the contents of the reaction flask was air cooled to 63-65° C. over 30 minutes. The solution was seeded pure ethylene diamine carboxylate crystals. After a few minutes crystallization began. The solution was cooled to room temperature then further chilled to 0° C. in an ice water bath for 1.0 h. The solid was isolated by filtration and the filter cake was rinsed with 2×2.0 ml isopropanol. The product was dried under Hi-vac at room temperature for 20 h or to constant weight.

Karl Fisher: 0.075 %; M. P.: 156-159° C.

¹H NMR was satisfactory.

DSC showed minor sharp endotherm at 81.46° C. and major sharp endotherm at 166.68° C. IR(KBr) was identical to the crude product.

Recrystallization was also done in 99/1 isopropanol/H₂O (91.0%), EtOAc/MeOH (74.8%) with similar quality of crystal.

### EXAMPLE 3

### Preparation of Tris(hydroxymethyl)aminomethane Salt

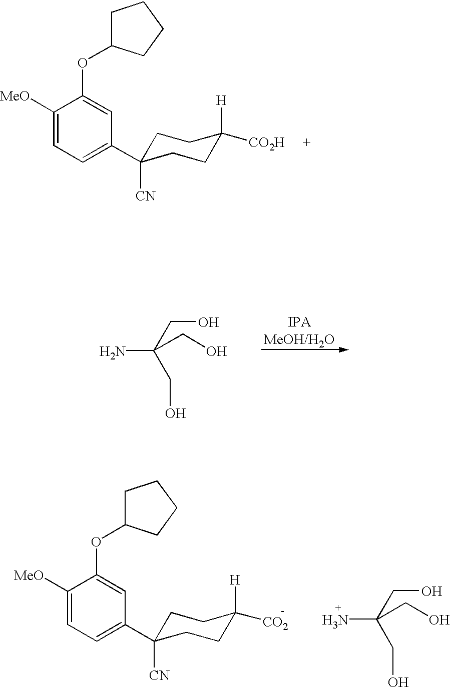

3(a) A 50 ml three neck round bottom flask was equipped magnetic stirrer, reflux condenser, addition funnel, thermometer and N₂ inlet. It was charged with 3.0 g (8.735 mmoles) of cis-4-cyano-4[3- (cyclopentyloxy)-4-methoxyphenyl]cyclohexane-1-carboxylic acid and 30 ml of isopropanol at room temperature. The mixture was heated to 70° C. with vigorous stirring to obtain a clear solution. While maintaining the reaction temperature at 70° C., tris(hydroxymethyl)aminomethane (1.10 g (9.091 mmoles, 1.04 equivalents) was added in a mixture of 8.0 ml of MeOH and 1.0 ml of deionized water. Some heating was required to obtain a clear solution of the amine. The resulting clear solution was stirred and cooled to room temperature for 2.0 hours without precipitation. Upon stirring overnight (16 hours) at room temperature, heavy precipitation formed. The slurry was diluted with 15 ml of isopropanol, then was transferred to a sintered funnel under vacuum. The filter cake was rinsed with 2×5.0 ml of cold isopropanol (0-5° C.). The product was dried under Hi-vac for 72 hours.

¹H NMR was satisfactory.

M.P.: 143-147° C.

Karl Fisher: 1.73%.

DSC had two sharp endotherm at 127.8° C., 140.9° C. and probable decomposition at 185° C.

IR(KBr pellet): 3513, 3327, 2954, 2865, 2233, 1590, 1519, 1409, 1197, 1258, 1244, 1197, 1168, 1147, 1122, 1095, 1063, 1048, 1028, 1002, 932, 806, 656, 635 cm−1.

3(b) Recrystallization: A 25 ml three neck round bottom flask equipped with magnetic stirrer, reflux condenser, addition funnel, thermometer and N₂ inlet was charged with 1.0 g of crude tris salt prepared in 3(a) and 7 ml of isopropanol at room temperature under N₂ atmosphere. The resulting mixture was heated to reflux under N₂ at 80-82° C. After approximately 20 minutes, the mixture became a clear yellow solution. The heating was removed and allowed to cool. Upon reaching 60° C. rapid precipitation was observed. An additional 2.0 ml of isopropanol was added to facilitate stirring. The solution was further cooled to room temperature over 45 minutes. The suspension was further chilled to 0-5° C. for 30 minutes in an ice water bath. The product was isolated by filtration through sintered funnel and the cake was rinsed with 2×2 ml of cold isopropanol 2×2.0 ml. The wet solid was dried under Hi-vac. for 24 hours yielding the tris salt of cis-4-cyano-4-[3- (cyclopentyloxy)-4-methoxyphenyl]cyclohexane-1-carboxylic acid.

¹H NMR was satisfactory.

M. P.:145-148° C.

Karl Fisher: 0.32%.

DSC indicated one sharp major endotherm at 147.1° C. and one shoulder at 153.2° C.

IR (KBr) was identical to the crude product.

Recrystallization was also done in EtOAc/isopropanol and acetone/isopropanol.

What is claimed is:

1. Sodium cis-4-cyano-4-[3-(cyclopentyloxy)-4-methoxyphenyl]cyclohexane-1-carboxylylate.

2. Ethylene diamine cis-4-cyano-4-[3- (cyclopentyloxy)-4-methoxyphenyl]cyclohexane-1-carboxylate.

3. Tris(hydroxymethyl)aminomethane cis-4-cyano-4-[3-(cyclopentyloxy)-4-methoxyphenyl]cyclohexane-1-carboxylate.
